(19) United States
(12) Patent Application Publication
(10) Pub. No.: US 2024/0018084 A1
(43) Pub. Date: Jan. 18, 2024

(54) Process for Removal of Fluoroorganic Compounds From Aqueous Media

(71) Applicant: 3M INNOVATIVE PROPERTIES COMPANY, St. Paul, MN (US)

(72) Inventors: Klaus Hintzer, Kastl (DE); Stefan Huber, Loiching (DE); Valentina Krupp, Waldkraiburg (DE); Herbert Koenigsmann, Burgkirchen (DE); Scott D. Schmick, Lake Elmo, MN (US); Zhongxing Zhang, Woodbury, MN (US); Michael D. Barrera, Oakdale, MN (US); William M. Lamanna, Stillwater, MN (US); Chetan P. Jariwala, Woodbury, MN (US); Markus Hirschberg, Mühldorf (DE)

(21) Appl. No.: 18/018,282

(22) PCT Filed: Jul. 30, 2021
(86) PCT No.: PCT/US2021/043958

§ 371 Date: Jan. 27, 2023

(30) Foreign Application Priority Data
Jul. 30, 2020 (EP) 20188786.6

(51) Int. Cl. C07C 51/48; C02F 1/26

(52) U.S. Cl. CPC C07C 51/48; C02F 1/26; C02F 2303/16; C02F 2101/36

(57) ABSTRACT

The present disclosure provides a process for removing fluoroorganic acidic compounds from a solution comprising at least one protic solvent, comprising: forming a mixture of a solution comprising at least one fluoroorganic acidic compound comprising less than 15 carbon atoms and protic solvent, wherein the solution has a pH of 4, with an extraction composition comprising at least one trialkylamine and organic solvent; reacting the fluoroorganic acidic compound with the trialkylamine to form a hydrophobic ionic compound comprising the anion of the fluoroorganic acidic compound and the cation of the trialkylamine; separating the mixture into a first phase comprising the protic solvent and at most 50 wt. % of the fluoroorganic acidic compound present in the initial solution; and a second phase comprising the organic solvent and hydrophobic ionic compound; removing the second phase from the first phase; and repeating steps aforementioned at least once more, wherein the first phase obtained in the last step is used as the initial solution.

### FIELD

The present disclosure relates to a continuous, multi-stage for removing fluoroorganic compounds from aqueous media by means of extraction with alkylamines in organic solvents.

### BACKGROUND

Fluorinated compositions have been used in a wide variety of applications including fluorochemicals for: water-proofing materials, fire-fighting foams for electrical and oil/grease fires, semi-conductor etching, and lubricants; and fluoropolymers for: hoses, gaskets, seals, coatings, and films. Reasons for such widespread use of fluorinated compositions include their favorable physical properties, which include chemical inertness, low coefficients of friction, and low polarizabilities (i.e., fluorophilicity), and high temperature stability.

After production of a fluorinated composition, fluorinated compounds, including, for example, starting materials and reaction by-products, may be removed from the waste streams generated by the production. The removal of the fluorinated compounds may be to recover expensive starting material and/or to avoid undesirable release of the fluorinated compounds into the environment.

Processes to remove or recover fluorinated compounds include ion exchange, ultrafiltration, distillation, liquid-liquid extraction, reverse osmosis, and adsorption on clays, carbon and other media. Such processes have been described in U.S. Publ. Nos. 2006/0205828 (Maurer et al.), 2007/0025902 (Hintzer et al.), and 2010/0084343 (Mader et al.), and U.S. Pat. No. 3,882,153 (Seki et al.), U.S. Pat. No. 4,369,266 (Kuhls, et al.), U.S. Pat. No. 6,642,415 (Fuhrer et al.), U.S. Pat. No. 7,279,522 (Dadalas et al.), and U.S. Pat. No. 5,603,812 (Hommeltoft). It is known that these processes may be associated with deficiencies of various degrees, such as low efficiency, high investment costs, or an increased environmental burden. Another more recent effort comprised the removal of fluoroorganic acids from aqueous media with alkylammonium compounds. However, providing of efficient and environmentally benign methods for removal of fluoroorganic compounds remains a desire in the art, in particular for large-scale or industrial operations.

### SUMMARY

The present disclosure provides a process for removing fluoroorganic acidic compounds from a solution comprising at least one protic solvent, comprising the following steps:
(i) forming a mixture of
   a. a solution comprising at least one fluoroorganic acidic compound comprising less than 15 carbon atoms and at least one protic solvent, wherein the solution has a pH-value of up to 4, with
   b. an extraction composition comprising at least one trialkylamine and at least one organic solvent;
(ii) reacting the fluoroorganic acidic compound with the trialkylamine to form a hydrophobic ionic compound comprising the anion of the fluoroorganic acidic compound and the cation of the trialkylamine;
(iii) separating the mixture into a first phase comprising the at least one protic solvent and no greater than 50% by weight of the total amount of the at least one fluoroorganic compound initially present in the solution in step (i); and a second phase comprising the at least one organic solvent and the hydrophobic ionic compound;
(iv) removing the second phase from the first phase; and
(v) repeating steps (i) to (iv) at least a second time, wherein the first phase obtained in step (iv) is used as the solution in step (i).

### DETAILED DESCRIPTION

Before any embodiments of this disclosure are explained in detail, it is to be understood that the disclosure is not limited in its application to the details of construction and the arrangement of components set forth in the following description. The invention is capable of other embodiments and of being practiced or of being carried out in various ways. As used herein, the term “a”, “an”, and “the” are used interchangeably and mean one or more; and “and/or” is used to indicate one or both stated cases may occur, for example A and/or B includes, (A and B) and (A or B). Also herein, recitation of ranges by endpoints includes all numbers subsumed within that range (e.g., 1 to 10 includes 1.4, 1.9, 2.33, 5.75, 9.98, etc.). Also herein, recitation of “at least one” includes all numbers of one and greater (e.g., at least 2, at least 4, at least 6, at least 8, at least 10, at least 25, at least 50, at least 100, etc.). Also, it is to be understood that the phraseology and terminology used herein is for the purpose of description and should not be regarded as limiting. Contrary to the use of “consisting”, which is meant to be limiting, the use of “including,” “containing”, “comprising,” or “having” and variations thereof is meant to be not limiting and to encompass the items listed thereafter as well as additional items. It should be noted, however, that the use of “comprising” herein also encompasses the term of “consisting of”, i.e. the use of “consisting of” in the sense of “consisting only of” is not excluded in the present disclosure per se.

Amounts of ingredients of a composition may be indicated by % by weight (or “% wt”. or “wt.-%”) unless specified otherwise. The amounts of all ingredients gives 100% wt unless specified otherwise. If the amounts of ingredients are identified by % mole the amount of all ingredients gives 100% mole unless specified otherwise.

Unless explicitly indicated, all preferred ranges and embodiments may be combined freely.

Parameters as described herein may be determined as described in detail in the experimental section.

The present disclosure provides a process for removing fluoroorganic acidic compounds from a solution comprising at least one protic solvent, comprising the following steps:
(i) forming a mixture of
   a. a solution comprising at least one fluoroorganic acidic compound comprising less than 15 carbon atoms and at least one protic solvent, wherein the solution has a pH-value of up to 4, with
   b. an extraction composition comprising at least one trialkylamine and at least one organic solvent;
(ii) reacting the fluoroorganic acidic compound with the trialkylamine to form a hydrophobic ionic compound comprising the anion of the fluoroorganic acidic compound and the cation of the trialkylamine;
(iii) separating the mixture into a first phase comprising the at least one protic solvent and no greater than 50% by weight of the total amount of the at least one fluoroorganic acidic compound initially present in the solution in step (i); and a second phase comprising the at least one organic solvent and the hydrophobic ionic compound;
(iv) removing the second phase from the first phase; and
(v) repeating steps (i) to (iv) at least a second time, wherein the first phase obtained in step (iv) is used as the solution in step (i).

With this process according to the present disclosure, an efficient removal of the fluoroorganic acidic compounds from solutions in protic solvents becomes feasible. The process according to the present disclosure is particularly advantageous for the removal of fluorinated acidic compounds from alcoholic and/or aqueous solvents or solvent mixtures on a large scale or in continuous, multi-stage processes. This is highly advantageous for treatment of large amounts of sewage as necessary during large-scale industrial manufacturing or laboratory sites. In this regard, it is believed that the fluoroorganic acidic compound reacts with the at least one trialkylamine to form a hydrophobic ionic compound comprising the fluoroorganic acidic anion and the trialkylammonium. This hydrophobic ionic compound then readily transfers from the protic solvent of the first solution into the organic solvent of the second solution. Accordingly, when separating the first (protic) phase from the (organic) second phase, an effective removal of a major part of the fluoroorganic acidic compounds initially present in the solution a. is achieved by the process according to the present disclosure. That means, that for extraction an extraction composition b. comprising at least one trialkylamine and at least one organic solvent is used.

Thus, in step (i) of the process according to the present disclosure, a mixture is formed of the solution a. and the extraction composition b. Forming a mixture includes providing solution a. and extraction composition b., bring both a. and b. into contact with each other, and may further include mixing, stirring, agitating or other steps well-known to the skilled person to form a mixture of two liquid components.

In step (ii), the reaction between the trialkylamine and the fluoroorganic acidic compound takes place, in which the hydrophobic ionic compound is formed. In this regard, “reacting” includes “allowing to react”, which means the case when both components get into contact, the reaction automatically takes place.

Further, in step (iii) of the process according to the present disclosure, the mixture is separated into a first phase comprising the at least one protic solvent and no greater than 50 wt.-% of the total amount of the fluoroorganic acidic compound initially present in the solution a. in step (i). Preferably, the fluoroorganic acidic compound is reduced by at least 20% in the first extraction stage compared to the initial concentration. Given the hydrophobic nature of the ionic compound formed in step (ii) and a generally desirable certain immiscibility of the at least one protic solvent and the at least one organic solvent, a separation of the mixture in the two phases as described herein may readily take place once mixing as described herein is stopped. This may include transfer of the mixture as described herein into another vessel or compartment. In general, and as known in the art, phase separation into the two phases as described herein may already be visible even upon inspection with the naked eye. However, determination and verification of phase separation may additionally carried out by means well-established in the art. Preferably, the part of total amount of the at least one fluoroorganic compound as described herein is at least 60 wt.-%, preferably at least 65 wt.-%, and more preferably at least 70 wt.-% of the total amount of fluoroorganic acidic compound initially present in the first solution in step (i). The major part as described herein may even be greater, such as at least 80 wt.-% or even at least 90 wt.-%, based on the total amount of fluoroorganic acidic compound initially present in the first solution in step (i).

Removing the second phase from the first phase in step (iv) may be then carried out by any common means known in the art to the skilled person. Preferably, those means and methods will be selected which are suitable for continuous and/or large-scale operations. Steps (i) to (iv) are repeated at least a second time, wherein the first phase obtained in step (iv) is used again as the solution a. in step (i). In this regard, the process may be carried out again with the first solution consisting essentially of the first phase obtained in step (iv). It is also possible to add the first phase into another fresh portion of first solution a. This may be the case when carrying out the process as described herein in a continuous, large scale operation mode. Accordingly, a more complete removal of the fluoroorganic acidic compound may be achieved.

Since wastewater comprising fluoroorganic compounds often are produced continuously during manufacturing in chemical industry, it is highly desirable that these by-product-containing wastewaters are cleaned from these compounds continuously and not batch-wise. Also, the process according to the present disclosure is highly suitable for continuous-mode operations. Accordingly, it is preferred that the process as described herein is carried out in a continuous mode. Preferably, this implies that there is a continuous supply of the solution comprising the at least one fluoroorganic acidic compound (a.) and/or extraction composition (b.) in step (i), a continuous reaction between the fluoroorganic acidic compound in step (ii), continuous separation in step (iii), and/or continuous removal second phase from the first phase in step (iv). Such a continuous mode of operation also preferably implies that each of the process steps as described herein are carried out in different vessels and/or compartments to which the product(s) of the previous product step is transferred into, e.g. by means of respective piping. It is also preferred that the process as described herein is carried out in a continuous mode in that that the second phase is continuously extracting the first phase in a multistage and continuous process.

The term “protic solvent” has the meaning commonly used in the art and describes a solvent whose molecules can either donate at least one proton to other molecules. Protic solvents are generally known to the skilled person and may either be selected according to the present needs of the process and the solubility of the fluoroorganic acidic compound. However, it may also occur that the protic solvent is already part of or even constitutes the major part of the wastewater and, therefore, cannot be selected. Preferably, at least one first protic solvent is selected from water, at least one alcohol, at least one acid, and any combinations and mixtures thereof. This has the advantage that a good phase separation between the first phase and the second phase in step (iii) of the process according to the present disclosure may be obtained, which is generally very desirable for a good removal of the second phase from the first phase in subsequent step (iv) and/or the time it takes to complete step (iii). In this regard, it is preferred that the at least one alcohol is selected from methanol, ethanol, propanol, butanol, pentanol, hexanol, and any combinations and mixtures thereof. The acid may be selected from organic acids such as formic acid and acetic acid and from inorganic acids such as HF, H₃PO₄, H₂SO₄, HCl, or HNO₃. In this regard, solutions or dilutions of these acids in water are preferably employed in the process as described herein. The water content of such acid-containing phases may be for example less than 99.9 wt.-%, less than 99 wt.-%, or even less than 98 wt.-%, based on the total weight of the first phase. On the other hand, the content of the alcohols can be up to 99 wt.-%, up to 95 wt.-%, or up to 90 wt.-%, based on the total weight of the first phase. The water content can be very low, e.g. lower than 70%, or 60% or even below 25 w % based on the total weight of the first phase. Hence, with the method according to the present disclosure it is possible to extract fluorinated acids from highly concentrated acids. The pH-value of the first phase may be up to 3.5, preferably up to 3, and more preferably up to 2.

The at least one fluoroorganic acidic compound may be selected from aliphatic linear, branched or cyclic fluorinated acidic compounds. In this regard, the fluorinated acids may be selected from fluorinated acids comprising a carbon backbone of at least 2 carbon atoms, preferably of at least 3 carbon atoms, for example, at least 5 carbon atoms, wherein the carbon backbone optionally contains at least one oxygen atoms. The fluorinated acids may be selected from fluorinated acids comprising a carbon backbone of 25 carbon atoms or less, preferably of 20 carbon atoms or less, and more preferably of 15 carbon atoms or less. It is particularly preferred that the fluorinated acids are selected from fluorinated acids comprising a carbon backbone of from 2 to 15 carbon atoms, preferably from 3 to 14 carbon atoms, and more preferably from 2 to 10 carbon atoms. The at least one fluorinated acidic compound may comprise at least one carboxylic acid moiety, at least one sulfonic acid moiety, at least one sulfate moiety, and/or at least one alcohol moiety. As known to the skilled person, fluorinated alcohols are acidic and fall under the definition of fluorinated acidic compounds as used herein and may therefore be advantageously removed by the process according to the present disclosure. The fluorinated acids comprise carboxylic acids (R_{f}—COOH), sulfonic acids (R_{f}—SO₃H), sulfates R_{f}—CH₂—O—SO₃⁻) , or alcohols (R_{f}—CH₂OH), dicarboxylic acids (HOOC—R_{f}—COOH), disulfuric acids (HO₃S—R_{f}—SO₃H).

The acids/alcohols can be perfluorinated or partially fluorinated. Examples are e.g. CF₃COOH, CF₂HCOOH, C₂F₅COOH, C₂F₄H—COOH, C₄F₉—COOH, C₇F₁₅COOH, CF₃—O—(CF₂)₃—O—CF₂COOH,

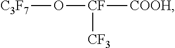

CF₃SO₃H, C₂F₅SO₃H, HOOC—(CF₂)ₙ—CF₂—SO₃H, C₂F₄H—SO₃H

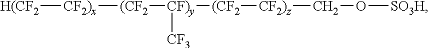

CF₃—CH₂OH,

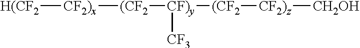

CF₃—O—(CF₂)₃—O—CFH—CF₂COOH, CF₃—O—(CF₂)₂COOH

The fluorinated acids can also have 1-4 hydrogen and/or chlorine within the molecule. Further examples are the commercially available compounds known under the designations OPA and ADONA (Ammonium 4,8dioxy-3H-perfluorononanoate).

In particular, some fluoroorganic compounds as described herein are known as PFCA's (perfluorinated carboxylic acids). Thus, removal of present commercially available fluoroorganic compounds become feasible with the process according to the present disclosure. In this regard, the fluoroorganic acidic compounds are fluorinated or perfluorinated C2 to C20 carboxylic acids. Typically, the fluoroorganic acidic compounds are present in the first solution in an amount of from 0.4 ppb to 30,000 ppm. Surprisingly, with the present invention fluorinated acidic compound can be extracted with a high selectivity from the hydrogen containing analogs from the aqueous phase (see examples).

With regard to the at least one alkylamine used in the processes according to the present disclosure, the at least one alkylamine may be selected from tertiary alkylamines, preferably from higher boiling trialkylamines, i.e. a trialkylamine having a high boiling point. The at least one trialkylamine comprises linear or branched alkyl groups; the alkylgroups comprising at least 4 carbon atoms, preferably at least 5 carbon atoms, and more preferably at least 6 carbon atoms. The at least one trialkylamine may comprise linear or branched alkyl groups comprising up to 40 carbon atoms, in total preferably up to 35 carbon atoms, and more preferably up to 30 carbon atoms. Accordingly, it is preferred that the at least one alkylamine comprises linear or branched alkyl groups comprising from 15 to 40 carbon atoms, preferably from 15 to 35 carbon atoms, and more preferably from 18 to 19 carbon atoms. For example, it is preferred that the at least one trialkylamine is a tertiary amine, i.e. a trialkylamine, wherein the sum of all carbon atoms is in the range of from 18 to 29. With the at least one alkylamine having carbon atoms in the preferred ranges as described herein, a particularly efficient removal of the fluoroorganic acidic compounds from the first solution as described herein may be achieved.

The amines of the present disclosure may be a liquid and should have a boiling point below 450° C. at room temperature. In one embodiment, the amine useful in the present disclose may be described as a water insoluble amine having a solubility in water of less than 20 mg, 10 mg, 5 mg, 3 mg, 2 mg, 1 mg, 0.75 mg, 0.5 mg, or even less than 0.25 mg per 100 mL when measured at ambient conditions.

Preferably, the at least one alkylamine is an amine according to an amine of formula (I):

NR₁R₂R₃ (I)

wherein R₁, R₂, and R₃ may be the same or different and at least one of R₁, R₂, and R₃ is a linear or branched, saturated or unsaturated carbon group, which optionally, may comprise heteroatoms. Preferably, any one of R₁, R₂ and R₃ comprise at least 4 carbon atoms, preferably at least 5 carbon atoms, and more preferably at least 6 carbon atoms. For example, the number of carbons in R₁-R₃ can vary, including, for example carbon backbones comprising least 5, 6, 7, 8, 10, 12, or even 14 carbon atoms. Preferably, the at least one alkylamine as used herein is selected from trihexylamine, trioctylamine, tridecylamine, tridocecylamine, alamine, triisooctylamine, trioctyl-decylamine, N-methyl-dioctylamine, N,N-dimethyloctylamine, tributylamine, octylamine, dioctylamine, and any combinations and mixtures thereof. High purity tertiary amines advantageous for use in the process according to the present disclosure are commercially available, for example, from Cognis, Monheim, Germany under the following trade designations: “ALAMINE 300” (tri-n-octylamine), “ALAMINE 308” (tri-isoctylamine), “ALAMINE 336” (tri(C₈/C₁₀) amine), “ALAMINE 310” (tri-isodecylamine), and “ALAMINE 304” (tri-laurylamine). Tertiary trialkylamines have the advantage over primary and secondary amines since these may undergo undesired side reactions with the fluorinated acids, e.g. by forming amides.

The temperature of the mixture, i.e. the aqueous emulsion after addition of the at least one alkylamine, may be controlled in order to advantageously enhance the removal of the fluoroorganic compounds in the process as described herein. In this regard, it is preferred that the process is conducted at a temperature of less than 80° C., preferably less than 70° C., more preferably of less than 60° C. Preferably, the process is conducted at a temperature in the range of from 3° C. to 80° C., preferably from 5° C. to 70° C., more preferably from 10 to 60° C., even more preferred from 15 to 50° C. In particular, the process as described herein may be carried out at room temperature and general ambient conditions. This is very advantageous for carrying out the process on an industrial scale since neither cooling nor heating is required, which greatly helps to save on energy, equipment and resources and makes the process as described herein very advantageous from both economic and ecologic points of view.

Generally, the at least one alkylamine is present in at least one organic solvent. This has the advantage that the extraction of the at least one fluoroorganic compound may be speed up. In this regard, it is preferred that the at least one organic solvent is not miscible with water. This has the effect that a clear separation between an aqueous phase and an organic phase may be obtained. Generally, the at least one organic solvent has a solubility in water at a temperature of 20° C. according to the method of the “Prüfmethoden-Verordnung (EG) Nr. 440/2008, Teil A, Methode A.6 (Test according to OECD-Prüfrichtlinie 105), of less than 500 ppm and more preferably less than 50 ppm. Furthermore, i the at least one organic solvent exhibits a boiling point of at least 50° C., preferably of at least 70° C., and more preferably of at least 100° C. The use of organic solvents exhibiting boiling points at ambient pressure above the aforementioned temperatures, i.e. high-boiling organic solvents, have the advantage of an improved inherent process safety in that the risk of solvent evaporating or even boiling off at the preferred temperatures of the process as described herein is greatly reduced. Preferably, the at least one organic solvent is selected from linear hydrocarbons, cyclic hydrocarbons, ethers, aromatic solvents, and any combinations and mixtures therefrom. For instance, toluene (boiling point 111° C.), mesitylene (boiling point 165° C.) or octane (boiling point 125° C.) may be used. In this regard, higher boiling points are preferred, i.e. mesitylene is preferred over octane. As an alternative or in addition to the beforementioned solvents, it is preferred to use fluorinated solvents, e.g. HFE's, Novec-Fluids (available from 3M). Such solvents preferably comprise perfluorinated trialkylamines (NR₃) and/or fluorinated ethers (e.g. Methoxyperfluorobutene HFE 7100). It is preferred that the boiling point of fluorinated solvents is higher than 50° C. The non-fluorinated solvent should have no reactive functional group, e.g. ester, ketone, an OH-group, since they might react with the fluorinated acid b. Furthermore, it is preferred that the volume ratio of solvent to amine is from 1:1 to 1000:1. In this regard, it is also preferred that the molar ratio of amine to fluorinated acid/alcohol is at least 1:1 up to 1000:1, preferably from 1:2 up to 100:1. These solvents may also be halogenated such as chlorinated or fluorinated, which is advantageous in extracting the fluorinated or perfluorinated compounds in the process according to the present disclosure. In this regard, it is preferred that the at least one organic solvent is selected from toluene, xylene, mesitylene, octane, or gasoline, and any combinations and mixtures or halogenated derivates therefrom.

Furthermore, it was found that further improved extraction results may be obtained when non-fluorinated polar organic co-solvents are used in combination with the aforementioned solvents. Preferably, compounds are used as non-fluorinated polar organic solvents which may also be characterized as surfactants. Accordingly, it is preferred that in addition to the solvents as described herein at least one non-fluorinated surfactant is used. A surfactant as used herein comprises a polar moiety and a non-polar moiety. For instance, the polar moiety may comprise an amide or heterocycle such as a pyrrolidinone, and the non-polar moiety may be a linear or branched alkyl group. The hydrophilic-lipophilic balance of these compounds as described herein may be estimated by means of the HLB value, as commonly determined by means of Griffin's method as follows:

HLB=20*M(H)/M

M(H) is the molecular weight of the hydrophilic portion of the molecule
M is the molecular mass of the whole molecule

For use herein, it is preferred that the at least one non-fluorinated surfactant exhibits a HLB value of 5 and less, preferably of 4 and less, and more preferably of 3 and less. Commercially available examples for the at least one non-fluorinated surfactant are the “Surfadone” compounds from Ashland Speciality Corp such as “Surfadone LP-300”. It is preferred that the at least one non-fluorinated surfactant is contained in the extraction composition b. in an amount of from 0.1 to 60 wt.-%, preferably 0.15-50 wt.-%, more preferably in an amount of 0.2-10 wt.-% based on the total weight of the extraction composition b.

Contacting the first solution with the second solution comprising the at least one trialkylamine in step (i) of the process as described herein, preferably present in at least one organic solvent as described herein, may be carried out by adding the second solution to or into the first. After addition, it is preferred that the resulting mixture is agitated for at least the amount of time sufficient to contact the at least one alkylamine with the fluoroorganic compound present in the first solution. As a general rule, however, the contact time between the at least one alkylamine, i.e. the second solution and the first solution is preferably in excess of 0.1 seconds with some equipment, but generally less than 5, 4, 3, or even 2 hours. Exemplary equipment that may be used for agitation include: vortexers, agitators, sonicators, and other such equipment as known in the art.

Without wanting to be bound by theory, it is believed that the extraction of the fluoroorganic acidic compounds, in particular acids, from the first solution of protic solvents with alkylamines as described herein, is effectuated with the fluoroorganic acid and the alkylamine forming an ion pair hydrophobic organic liquid. This would then separate from the protic solvent of the first solution and allow subsequent for further extraction and treatment. This separation is even enhanced when using the least one organic solvent in the second solution as described herein when the first solution is contacted with the at least one alkylamine in the process according to the present disclosure. Because of this immiscibility, the first phase (i.e. protic) phase and the second phase (i.e. organic phase or non-protic phase) generally separate after agitation has stopped. If, however, the mixture, although bi-phasic, does not phase separate on its own, measures as known in the art, may be used to promote the phase separation, including centrifugation. Separation of the first phase from the second phase in step (iv) of the process as described herein may be carried out by any appropriate means known in the art. Surprisingly, by the process according to the present disclosure, fluorinated acids can be separated from other non-fluorinated acids (e.g. CH₃COOH vs. CF₃COOH). Thus, the solution a. in the process described herein may further comprise at least one inorganic acid, preferably selected from sulfuric acid, hydrochloric acid, acetic acid, nitric acid, and any combinations and mixtures thereof.

The process according to the present disclosure is preferably carried out in a continuous mode. This is very advantageous for applications in industrial scale, where e.g. continuous treatment of wastewater is necessary. Preferably, the process as described herein is carried out in extraction columns, preferably continuous multi-staged extraction columns. In this regard, it is preferred that multi-staged extraction columns as known in the art comprise at least two stages. Preferably, the process is carried out in Scheibel-columns, centrifugal extractors (e.g. from Robatel), and other means known to the skilled person in the art. Such devices are described in Chem. Ing. Tech. 2020, 92, No. 12, 1941-1952.

It was further found that the processes according to the present disclosure show best results when carried out at certain pH-values. In particular, it was found that the best results were obtained at low pH-values, while medium pH-values showed only mediocre results and high pH-values failed to achieve effective removal of fluoroorganic compounds. Running the process as described herein at the preferred pH-values may be easily achieved by adjusting the pH-value of the first solution (a.) before contacting it with the at least one alkylamine. Accordingly, it is preferred that the pH-value of the first solution is adjusted to a pH-value of up to 4, preferably of up to 3, more preferably of up to 2 before contacting with at least one alkylamine in step (i). Also, this regeneration process is preferably carried out as continuous, multistage process.

In order to run a process in the chemical industry in an economically efficient way and in order to comply with environmental benign requirements, there is a desire in the art to recollect and, in if feasible, recycle or reuse the material and compounds used in the process. In the process according to the present disclosure, it is desirable to at least recollect the at least one alkylamine used in the process. It was found that this may be achieved by adding at least one base to the organic phase after carrying out the previously described process steps. Hence, it is preferred that the process according to the present disclosure comprises an additional step (vi) of adding at least one base to the organic phase in order to regenerate the at least one trialkylamine and/or solvent. Carrying out this additional step has the advantage that the at least one trialkylamine from its protonated form and/or solvent is recovered and can be recollected and/or recycled into the processes as described herein. Therefore, it is preferred that step (vi) is carried out continuously. It is further preferred that the regenerated at least one trialkylamine is recycled into use in step (i) of the process as described herein. Adding a base and recollecting the regenerated alkylamine may be carried out by common means known to the skilled person. With regard to the base, it is preferred that the at least one base is selected from KOH, NH₄OH, NaOH, and any combinations and mixtures thereof. These bases provide favourable results, dissolve readily in aqueous media, and are commercially available in any given amounts at reasonable prices. The pH of the basic, aqueous regeneration solution for the organic phase is may be above 8, preferably above 9 and more preferably above 10.

The regeneration of the organic phase to recover the trialkylamine and solvent—which means a clean, reusable extraction system—is preferably done via a multi-stage and continuous mode. By doing this, the clean extraction mixture can be fed directly into the aqueous phase for extracting the fluoroorganic acidic compound. Accordingly, aclosed loop system is obtained for the extraction of fluorinated acidic compounds from solutions, in particular aqueous solutions such as wastewater streams.

The extracted aqueous (1^{st} phase) phase may contain traces of the trialkylamine and/or solvent. Within the present process, it may be an option that such impurities are removed by treating the extracted aqueous phase with adsorbents, e.g. silica, activated carbon, alumina, mol-sieves etc. The treatment can be done batch-wise or continuously, e.g. using colums filled with adsorbents and running the aqueous phase through it; alternatively, the adsorbents can be put into the aqueous phase directly and filtered off after some time.

The fluorinated acidic compounds, which may be obtained in a highly concentrated from after carrying out the methods as described herein may be further treated, e.g. recovery of usable materials such as emulsifiers or destroyed e.g. by incineration or oxidation.

The process according to the present disclosure is preferably a continuous, multistage process for extracting fluoroorganic acidic compounds from a solvent stream, preferably a wastewater stream. The water stream is preferably selected from an industry sewage water stream, or a water stream from a vessel comprising a solvent phase containing at least one fluoroorganic acidic compound.

### EXAMPLES

The present disclosure is further described without however wanting to limit the disclosure thereto. The following examples are provided to illustrate certain embodiments but are not meant to be limited in any way. Prior to that some test methods used to characterize materials and their properties will be described. All parts and percentages are by weight unless otherwise indicated.

### Test Methods

### NMR Analytical Method

All of the FT-NMR spectra were acquired on Bruker AVANCE NEO 600 MHz and Bruker AVANCE III HD 600 MHz NMR spectrometers that were respectively equipped with a 5 mm SmartProbe and a 5 mm TCI Cryoprobe at ˜25° C. The TCI cryprobe was used for acquisition of ¹⁹F-NMR spectra for samples for select low level FC components.

### (1) Water Phase Samples:

For each sample, an aliquot of fluid was accurately weighed into a 5 mm o.d. glass NMR tube, was spiked with small accurately weighed amount of 2,2,2-trifluoroethanol internal standard, and was diluted with a small amount of deuterium oxide (D₂O) for NMR analyses. 600 MHz ¹H-NMR and 565 MHz ¹⁹F-NMR spectra were acquired. The trifluoroethanol was used in all cases as the internal standard to permit the calculation of the absolute weight percent concentrations of sample components without needing to identity and quantify all of the other components in the samples.

### (2) Organic Phase Samples:

For each sample, an aliquot of fluid was accurately weighed into a 5 mm o.d. glass NMR tube, was spiked with small accurately weighed amount of 1,4-bis(trifluoromethyl)benzene (p-HFX) internal standard, and was diluted with a small amount of deuterium acetonitrile (CD₃CN) for NMR analyses. 600 MHz ¹H-NMR and 565 MHz ¹⁹F-NMR spectra were acquired. The p-HFX was used in all cases as the internal standard to permit the calculation of the absolute weight percent concentrations of sample components without needing to identity and quantify all of the other components in the samples.

### Gas Chromatography Analysis

1 mL of the aqueous sample and 0.6 mL of BF₃/MeOH-complex solution (20%, obtained from Merck) are filled in 10 mL head-space vial and intermediately closed. After shaking, the vial is heated to 70° C. for 30 min using the autosampler unit of the headspace GC-instrument and afterwards analyzed by headspace-GC-FID with external calibration.
Instrument: Agilent 6890N
Column: Rtx-200, Trifluoropropylmethylpolysiloxane, 0.32 mm×105 m, 1.5 μm
Injection: 1500 μL
Temp: 50° C. (5 Min)-5° C./Min−>200° C.
Flow Rate: 3.5 mL/min

### Liquid Chromatography-Mass Spectrometry (LC-MS)

The submitted samples were dissolved in methanol prior to analysis. Standard solutions were prepared from an aqueous solution of Adona that was determined to have a concentration of 28.36% by NMR.
Instrument: Agilent 6470 Triple Quad LCMS MAID 1665
Column: Zorbax Bonus RP, 4.6×75 mm, 3.5 μm
Solvent A: Water with 6 mM ammonium acetate
Solvent B: Methanol with 6 mM ammonium acetate
Gradient: 75% B to 100% B at 4 minutes
Injection: 2 μL
Col. Temp: 40° C.
Flow Rate: 0.5 mL/min
MS: Negative electrospray

### Materials Used

TABLE 1
Materials used.

| | |
|---|---|
| Trioctylamine | obtained from Sigma Aldrich (99.7% purity) |
| Trifluoroacetic acid | Obtained from Sigma Aldrich (97% purity) |
| Pentafluoropropionic acid | Obtained from Sigma Aldrich (97% purity) |
| Tetrafluoropropionic acid | Obtained from abcr (94% purity) |
| Alamine 336 | Obtained from Cognis (>95% purity) |
| Mesitylene | Obtained from Sigma Aldrich (98% purity) |
| Octane | Obtained from Sigma Aldrich (98% purity) |
| KOH (Potassium hydroxide) | Obtained from Sigma Aldrich (>85% purity) |
| ADONA | Emulsifier, chemical formula CF3O—(CF2)3—O—CHF—CF2—COONH4, |
| | available from Anles Ltd., St. Petersburg, Russia |
| AOPA | emulsifier, chemical formula CF3—O—(CF2)2—COONH4, prepared as |
| | described in U.S. Pat. No. 6,482,979 A1 |
| LP-300 | Surfactant obtained from Ashland (N-dodecyl-2-pyrrolidone) having |
| | an HLB value of 3. |
| Activated Aluminium Oxide Basic | Obtained from Dynamic Adsorbents, Inc.(USA) |
| (non-fibrous) | |
| Granular Activated Carbon | Obtained from Carbot Corporation (USA) |
| Amorphous Silica Gel (SiO2) | Obtained from Multisob Filtration Group (USA) |

### Example 1

Aqeuous mixtures with different fluoroorganic acidic compounds were extracted with a mixture of 0.5 w % Alamine in mesitylene at different pH-values of the of the aqueous phase. The extractions were done at 20° C. in a single-stage to demonstrate the different effect of pH.

TABLE 2
Results of example 1.

| Initial concentration of | pH prior extraction | | |
|---|---|---|---|
| fluoroorganic acidic | pH 7 | pH 3 | pH 1.5 |
| compound in water (w %) | Concentration after extraction (w %) | | |
| CF₃—CO₂H (0.0329) | 0.0329 | 0.0043 | 0.0023 |
| HCF₂—CF₂—CO₂H (0.0111) | 0.011 | 0.00085 | 0.00062 |
| CF₃—CHF—CO₂H (0.0230) | 0.023 | 0.0058 | 0.0035 |

| | pH prior extraction | | |
|---|---|---|---|
| | pH 7 | pH 6 | pH 1.5 |
| CF₃—O—(CF₂)₃—O—CFH—CF₂—CO₂H (0.0399) | 0.0248 | 0.0258 | n.d. |
| CF₃—O—(CF₂)₂—CO₂H (0.00021) | 0.0002 | 0.0002 | n.d. |

n.d. = non detectable

The concentration of the fluoroorganic acidic compounds before and after extraction were determined by NMR.

### Example 2 (Comparative Example)

200 mL of an aqueous solution containing 328 ppm trifluoroacetic acid (TFA) and 38 ppm pentafluoropropionic acid was filled in a 250 mL plastic bottle. After adjusting the pH value of the aqueous solution to the target value (pH 3) with sulfuric acid, the extracting agent solution (1 g Alamine 336 dissolved in 19 g mesitylene) was added. Upon tightly sealing the bottle, the obtained mixture was vigorously shaken for 1 minute. After shaking the solution at 20° C., the the aqueous phase was separated from the extracting agent solution by filling the mixture in a separation funnel for phase splitting. NMR on the aqueous phase revealed a significant reduction of TFA down to 27 ppm and pentafluoropropionic acid <1 ppm. during the single extraction stage.

### Example 3

The extraction described in example 2 was repeated 4 times (4 stage extraction) on the similar aqueous solution as in example 2 in the following procedure. After each extraction step (1^{st} extraction similar procedure as example 2), the aqueous phase was separated from the extracting agent (1 g Alamine in 19 g mesitylene) and filled in a new 250 mL plastic bottle. Then a fresh extracting agent solution (1 g Alamine in 19 g mesitylene) is added, the mixture is shaken and again, the aqueous phase was separated. This procedure was repeated 3 times, so that the aqueous solution was purified in 4 stages (extraction steps). NMR on the aqueous solution after four extraction steps revealed a drastic reduction of TFA down to 0.3 ppm and pentafluoropropionic acid <1 ppm.

### Example 4

An aqueous solution comprising TFA, pentafluoro propionic acid and tetrafluoro propionic acid was treated as described in example 3 (a 4-stage batch extraction) was followed. The experiments demonstrate the benefit of the multistage extraction and the differences in solvents (Mesitylene versus Octane). The results are listed below:

TABLE 3
Results of example 4.

| | initial | Mesitylene | | | Octane | |
|---|---|---|---|---|---|---|
| | conc. | 1. stage | 2. stage | 4. stage | 2. stage | 4. stage |
| | ppm | ppm | ppm | ppm | ppm | ppm |
| CF₃—CO₂ | 330 | 43 | 5 | 0.3 | 50 | 14 |
| CF₃CFH—CO₂ | 230 | 58 | 12 | <1 | 73 | 29 |
| CF₃CF₂—CO₂ | 38 | <1 | <1 | <1 | <1 | <1 |

### Example 5

A 4-stage centrifugal extractor LX-204 from Rousselet Robatel (France) was used to purify a water stream with different fluorinated species listed in the table below.
Parameters: Phase ratio Alamine (5%) in Mesitylene vs. water=0.1 w/w;
   flow rate: 4.1 kg/min water phase, 0.41 kg/min organic phase; RPM=3300; RT; pH of water phase: 1.7.

TABLE 4
Results of example 5.

| | clear phase splitting | | |
|---|---|---|---|
| | | 4. stage | |
| | | extraction | |
| | initial | (aqueous | Organic |
| | conc. | phase) | phase |
| | ppm | ppm | ppm |
| —CF₃—CO₂ | 128 | <1 | 1130 |
| —CF₃CFH—CO₂ | 114 | <2 | 791 |
| —CF₃CF₂—CO₂ | 32 | <2 | 265 |

### Regeneration of Organic Phase

The collected organic phase was treated in the same apparatus LX-204 with 20 w % KOH solution at 20° C.
Parameters: Phase Ratio Organic/aqueous KOH=20 w/w.
   flow rates: 0.09 kg/min aqueous KOH phase, 1.82 kg/min organic phase, RPM 3000.
Results: All fluorinated species in the organic phase were below detection limit (<1 ppm). The organic Alamine/Mesitylene phase can be reused for further extractions.

### Example 6

A waterstream containing TFA was treated with a continuous, 4-stage-extraction (trioctylamine/Mesitylene) and additionally the organic phase was regenerated also with a continuous, 4 stages (18 w % NaOH).

Particles in wastewater were first filtered off with 1 μm filters. After pH adjusting to 1-2.0 with conc. sulfuric acid (96 w %), 100 kg/h wastewater containing 93 ppm C2 was extracted with 10.8 kg/h organic solvent (95% mesitylene and 5% trioctylamine) to remove the acids in the extraction (1. Centrifuge extractor from Robatel, 4 stages). After extraction, the TFA-content was <3 ppm (*Detection-limit).

The TFA loaded organic phase was regenerated by extraction with aqueous sodium hydroxide (18 w %) in the regeneration step (2. Centrifuge extractor, 3 stages) and fed back into the wastewater purification (1. Centrifuge extractor).

TABLE 5
Set parameters and levels for liquid-liquid extraction in example 6.

| | |
|---|---|
| Wastewater purification: | |
| wastewater flow rate [l/h] | 100 |
| H2SO4 96% feed [l/h] | 0.3 |
| Organic (Ms/Al) extraction phase [kg/h] | 10 |
| Rpm wastewater purification (1. Centrifuge) | 2400 |
| Regeneration of organic phase: | |
| Feed of sodium hydroxide solution18% [kg/h] | 1.0 |
| Feed of organic phase [L/h] | 10 |
| Rpm Regenaration (2. Centrifuge) | 2000 |
| TFA in wastewater [ppm] | 93 |
| TFA in wastewater after extraction [ppm] | <3 (DL*) |
| TFA in NaOH (aq. 18 w %) [w %] | 1.0 |
| Trial time | 14 days |

DL = detection limit

### Liquid-Liquid-Extraction (Mass Balance)

### 1. Centrifuge 2. Centrifuge

1^{st} centrifuge:

100 l/h heavy phase containing wastewater and 10 kg/h light phase containing Mesitylene/Alamine extraction agent are providing 100 L/h cleaned extracted water phase which is removed and 10 kg/h loaded light phase containing a mixture of Mesitylene/Alamine & fluoroorganic acidic compounds. Those 10 kg/h of loaded light phase is introduced into a second centrifuge and is regenerated with 1 kg/h NaOH, which gives 10 kg/h of regenerated mixture of light phase Mesitylene/Alamine and 1 kg/h heavy phase containing all fluoroorganic acidic compounds. The 10 kg/h regenerated light phase goes back into the extraction process in centrifuge 1.

This demonstrates the benefit of continuous, multistage extractions of contaminated water streams incl. the continuous, multistage regeneration to recover trialkylamine/solvent and establishing a closed loop.

### Example 7

An aqueous mixture comprising 3 wt.-% CF₃COOH, 65 wt.-% acetic acid, 10 wt.-% HF, 2.5 wt.-% HNO₃ was extracted with Alamine/Mesitylene as described in Example 3 with the exception that the Alamine/CF₃COOH ratio was varied. In sample 2, the initial 3 wt.-% CF₃COOH solution was diluted 1:1 with deionized water.

TABLE 5
Results of example 7.

| | | CF₃COO | CF₃COO | |
|---|---|---|---|---|
| Initial | Trioctylamine/ | concentration | concentration | |
| CF₃COO-conc. | CF₃COO-Ratio | after 1^{st} extraction | after 4 extractions | |
| 3 wt.-% | 1.6 | 0.9 wt.-% | 0.04 | wt.-% |
| 1.5 wt.-% | 2.6 | 0.2 wt.-% | 10 | ppm |

### Example 8

105 g of an aqueous mixture comprising 8.3 w % TFA, 65 w % CH₃COOH, 9 w % HF and 3 w % HNO₃ was extracted under vigorous stirring with 105 g of an extraction mixture containing 35 g trioctylamine and 70 g mesitylene. After phase separation NMR-measurement provided the following results.

TABLE 7
Results of example 8.

| | untreated mixture | extracted mixture |
|---|---|---|
| Content of CF₃COOH in | 8.3 w % | 0.8 wt.-% |
| aqueous mixture | | |
| Content of CH₃COOH in | 65 w % | 41 wt.-% |
| aqueous mixture | | |

The results from Example 7 and 8 show an extremely selective removal of TFA versus CH₃COOH from the aqueous mixture; furthermore, it demonstrates that fluorinated acids can be extracted from highly acidic media and low water contents.

### Example 9

A flask (250 mL) equipped with a magnetic stirr bar was charged at RT with an aqueous solution of HCl (30% w/w; 200 g) containing trifluoroacetic acid (237 μg). Then, the solution was extracted with a mixture consisting of trioctylamine and mesitylene (1 g trioctylamine+19 g mesitylene;). After phase, the lower phase was extracted again. The extraction process was conducted in the same manner as described before. After the corresponding phase separation, the lower phase was passed through a silica gel (0.1 wt %) column from the bottom up The results are summarized in table 8.

TABLE 8
Result of GC-analysis of example 110.

| | Untreated | | | |
|---|---|---|---|---|
| | solution | 1. Extraction | 2. Extraction | Silicagel |
| Content of | 237 μg | 66 μg | 29 μg | 19 μg |
| CF₃CO₂H | | | | |
| in HCl (30%) | | | | |

### Example 10

In order to demonstrate the effect of the non-fluorinated surfactant in the first extraction step of the method as described herein, in a plastic bottle, added 100 g on an aqueous mixture containing different species of fluroorganic acidic compounds which was adjusted to pH=1.7 using sulfuric acid. Then 10 g Mesitylene, 5 g LP-300, and 0.5 g Alamine were added to the bottle before the bottle was closed and shaken vigorously for 1 minute. After allowed to settle for 30 minutes, two liquid phases appeared in the bottle. A small sample was obtained from the bottom aqueous phase of the bottle and analyzed for fluorochemical contents by means of NMR analysis.

The procedure above was followed, except that no LP-300 was used. The results of example 10 and comparative example 2 are summarized in table 7.

TABLE 7
Results of example 10

| | | Extraction | |
|---|---|---|---|
| | | using | Extraction |
| | | mesitylene/ | using |
| | initial | LP-300/ | mesitylene/ |
| Fluoroorganic acidic | concentration | Alamine | Alamine |
| Compounds in water | [%] | [%] | [%] |
| 1. CF₃—CO₂⁽⁻⁾ | 0.0321 | 0.0023 | 0.0023 |
| 2. HCF₂CF₂—CO₂⁽⁻⁾ | 0.0113 | 0.00054 | 0.00062 |
| 3. CF₃CFH—CO₂⁽⁻⁾ | 0.0228 | 0.0028 | 0.0035 |
| 4. H—CF₂—CO₂⁽⁻⁾ | 0.0016 | 0.00078 | 0.00098 |

1. A process for removing fluoroorganic acidic compounds from a solution comprising at least one protic solvent, comprising the following steps:
(i) forming a mixture of
a. a solution comprising at least one fluoroorganic acidic compound comprising less than 15 carbon atoms and at least one protic solvent, wherein the solution has a pH-value of up to 4, with
b. an extraction composition comprising at least one trialkylamine and at least one organic solvent;
(ii) reacting the fluoroorganic acidic compound with the trialkylamine to form a hydrophobic ionic compound comprising the anion of the fluoroorganic acidic compound and the cation of the trialkylamine;
(iii) separating the mixture into a first phase comprising the at least one protic solvent and no greater than 50% by weight of the total amount of the at least one fluoroorganic acidic compound initially present in the solution in step (i); and a second phase comprising the at least one organic solvent and the hydrophobic ionic compound;
(iv) removing the second phase from the first phase; and
(v) repeating steps (i) to (iv) at least a second time, wherein the first phase obtained in step (iv) is used as the solution in step (i).

2. The process according to claim 1, wherein the process is carried out in a continuous operation mode.

3. The process according to claim 1 or claim 2, wherein the process is carried out in a continuous operation mode in that the second phase is continuously extracting the first phase in a multistage continuous process.

4. The process according to any one of the preceding claims, wherein the at least one first protic solvent is selected from water, at least one alcohol, at least one acid, and any combinations and mixtures thereof.

5. The process according to any one of the preceding claims, wherein the pH-value of the first phase is up to 3.5, preferably up to 3, and more preferably up to 2.

6. The process according to any one of the preceding claims, wherein the at least one fluoroorganic acidic compound is selected from aliphatic linear, branched or cyclic fluorinated acidic compounds, preferably wherein the at least one fluorinated acidic compounds comprises at least one carboxylic acid moiety, at least one sulfonic acid moiety, at least one sulfate moiety, and/or at least one alcohol moiety.

7. The process according to any one of the preceding claims, wherein the at least one alkylamine is selected from tertiary alkylamines, preferably wherein the at least one trialkylamine comprises linear or branched alkyl groups comprising at least 5 carbon atoms, preferably at least 6 carbon atoms, and more preferably at least 7 carbon atoms.

8. The process according to any one of the preceding claims, wherein the at least one trialkylamine is an amine according to an amine of formula (I):
NR₁R₂R₃ (I)
wherein R₁, R₂, and R₃ may be the same or different and at least one of R₁, R₂, and R₃ is a linear or branched, saturated or unsaturated carbon group, which optionally, may comprise heteroatoms.

9. The process according to any one of the preceding claims, wherein the major part of total amount of the at least one fluoroorganic acidic compound is at least 60 wt.-%, preferably at least 65 wt.-%, and more preferably at least 70 wt.-% of the total amount of fluoroorganic acidic compound initially present in the solution a. in step (i).

10. The process according to any one of the preceding claims, wherein the at least one organic solvent has a solubility in water at 20° C. of less than 0.1%, preferably less than 500 ppm and more preferably less than 50 ppm.

11. The process according to any one of the preceding claims, wherein the extraction composition b. further comprises at least one non-fluorinated surfactant preferably having a HLB value of 5 and less, preferably of 4 and less, and more preferably of 3 and less.

12. The process according to any one of the preceding claims, wherein solution a. further comprises at least one inorganic acid, preferably selected from sulfuric acid, hydrochloric acid, acetic acid, nitric acid, and any combinations and mixtures thereof.

13. The process according to any one of the preceding claims, wherein the process is carried out in extraction columns, preferably multi-staged extraction columns, Scheibel columns, centrifugal extractors, and any combinations thereof.

14. The process according to any one of the preceding claims, wherein the process comprises an additional step (vi) of adding at least one base to the organic phase in order to regenerate the at least one alkylamine.

15. The process according to claim 14, wherein step (vi) is carried out continuously and/or as a multistage process.

16. The process according to claim 14 or claim 15, wherein the regenerated at least one alkylamine is recycled into use in step (i).

17. The process according to any one of the preceding claims, wherein the process is a continuous process for extracting fluorinated compounds from a solvent stream, preferably a water stream.
